(19) United States
(12) Patent Application Publication
(10) Pub. No.: US 2024/0335371 A1
(43) Pub. Date: Oct. 10, 2024

(54) MAGNESIUM SULFATE AND FORSKOLIN FOR TREATMENT OF SEXUAL DISORDERS AND BENIGN PROSTATIC HYPERPLASIA

(71) Applicant: State-of-the-Art Technologies, Inc., Hawthorne, CA (US)

(72) Inventor: Sota Osemwota Omoigui, Hawthorne, CA (US)

(21) Appl. No.: 18/296,940

(22) Filed: Apr. 6, 2023

(51) Int. Cl. A61K 9/00; A61K 9/107; A61K 31/352; A61K 33/06; A61K 36/53; A61K 47/14; A61K 47/34

(52) U.S. Cl. CPC A61K 9/0034; A61K 9/0014; A61K 9/107; A61K 31/352; A61K 33/06; A61K 36/53; A61K 47/14; A61K 47/34

(57) ABSTRACT

Compositions and methods include magnesium sulfate or forskolin to treat erectile dysfunction, premature ejaculation, decreased libido, and benign prostatic hyperplasia in the male and orgasmic sexual dysfunction/decreased libido in the female. The compositions may be delivered topically or transdermally on the penis to reduce the symptoms of erectile dysfunction, premature ejaculation, low libido, by increasing blood flow to the penis, delivered topically in the ano-rectal or prostate region to reduce the symptoms of BPH by smooth muscle relaxation of the prostate, or by topical or transdermal application to the female genitalia, nipple and/or ano-rectal region, to treat low libido/orgasmic sexual dysfunction.

### TECHNICAL FIELD

The present disclosure relates generally to methods and compositions for treating various sexual disorders and benign prostatic hyperplasia (BPH), and more particularly to methods and compositions that include the administration of magnesium sulfate or forskolin to a male or female patient for treatment of sexual disorders or BPH.

### BACKGROUND

Erectile dysfunction (ED) is one form of impotence and a common medical problem. Impotence can be defined as a lack of power, in the male, to copulate and can involve an inability to achieve or maintain an erection sufficient for satisfactory sexual performance and/or ejaculation. The incidence of ED increases with age, with about 50% of men over the age of 40 suffering from some degree of ED. ED is attributed to organic (58%; generally not characterized, but including diabetes and excluding spinal cord injury), psychogenic (17%), or mixed (24%) etiologies.

The substance of the corpora cavernosa (erectile tissue of the penis) consists of numerous sinusoids (lacunar spaces) among interwoven trabeculae of smooth muscles and supporting connective tissue. The physiologic mechanism of erection of the penis involves release of nitric oxide (NO) in the corpus cavernosum during sexual stimulation. NO then activates the enzyme guanylate cyclase, which results in increased levels of cyclic guanosine monophosphate (cGMP), producing smooth muscle relaxation in the corpus cavernosum and allowing inflow of blood. There is increased blood flow into the expanding sinusoids of the corpora cavernosa, with the average volume increase of the erect penis from the flaccid volume of 3-fold with a range from 1.7-5 fold.

In females, low libido/orgasmic sexual dysfunction is defined as decreased arousal with deficient or absent sexual fantasies and desire for sexual activity. The clitoris is a nub of spongy tissue at the front of the vagina. Recent research reveals that much of the clitoris is internal, having 4-inch roots that reach into the vagina. When sexually aroused, it fills with blood, and the bundle of nerves in the tissue becomes sensitive to touch. Clitoral atrophy occurs when the clitoris stops responding to sexual arousal and no longer functions as it should. The clitoris can even disappear. This may be the result of inadequate blood flow to the vagina and clitoris and may also occur when the testosterone hormone level drops. Testosterone is responsible for libido. The sponge like tissue in a clitoris needs the hormone for proper arousal.

New onset of low libido can be a symptom of an underlying physical or mental health problem. A drop in libido can also be related to a life stage or event like pregnancy, post-partum, or grief due to a loss. A woman might find that her libido is heavily influenced by her partner's interest in or drive for sex.

Benign prostatic hyperplasia (BPH) is a condition in which the prostate gland becomes very enlarged and may cause problems associated with urination. One of the factors that contributes to BPH is the proliferation of smooth muscle tissue within the prostate gland. Smooth muscle is a type of involuntary muscle that contracts and relaxes in response to various stimuli. Smooth muscle tone is the degree of tension or contraction in the smooth muscle. Smooth muscle tone in the prostate gland depends on extra-and intracellular calcium stores and is regulated by various second messenger pathways, such as cyclic adenosine monophosphate (cAMP) and alpha-1 receptors. Alpha-1 receptors are a type of protein that bind to adrenaline and noradrenaline, which are hormones that stimulate the sympathetic nervous system. When alpha-1 receptors are activated, they increase the intracellular calcium levels and cause smooth muscle contraction. This can lead to increased prostatic smooth muscle tone and obstruction of the urinary flow.

### PRIOR TREATMENTS

Prior treatments for impotence target the non-adrenergic, noncholinergic L-arginine-NO pathway that is involved physiologically in mediating penile erection. For example, sildenafil citrate, one of the most popular treatment for impotence, belongs to a group of selective inhibitors of cGMP-specific phosphodiesterase type 5 (PDE5). It has no direct relaxant effect on isolated human corpus cavernosum, but enhances the effect of NO by inhibiting PDE5, which is responsible for degradation of cGMP in the corpus cavernosum. When sexual stimulation causes local release of NO, inhibition of PDE5 by sildenafil causes increased levels of cGMP in the corpus cavernosum, resulting in smooth muscle relaxation and inflow of blood to the corpus cavernosum.

Sildenafil citrate is administered orally (https://www.accessdata.fda.gov/drugsatfda_docs/label/2010/020895s033lbl.pdf). Maximum observed plasma concentrations are reached within 30 to 120 minutes (median 60 minutes) of oral dosing. The effect lasts for 4 hours with a diminishing response after 2 hrs. Systemic side effects include decreases in sitting blood pressure, most notably approximately 1-2 hours after dosing, and transient dose-related impairment of color discrimination (blue/green). Prolonged erection greater than 4 hours and priapism (painful erections greater than 6 hours in duration) have also been reported. Caution is advised when PDE5 inhibitors are co-administered with alpha-blockers. Both drugs are vasodilators with blood pressure lowering effects. In some patients, concomitant use of these two drug classes can lower blood pressure significantly leading to symptomatic hypotension (e.g., dizziness, lightheadedness, fainting).

Many compounds have been investigated for their therapeutic potential in the treatment of ED, including phenoxybenzamine, papaverine, prostaglandin E1 (PGE1), and phentolamine. These compounds, either alone or in combination, are typically self-administered by intracavernosal (i.c.) injection. While such treatments are effective, a treatment that is less invasive than injection therapy is preferred because pain, priapism, and fibrosis of the penis are associated with the i.c. administration of these agents.

For example, alprostadil (i.e., prostaglandin E1) delivered by intraurethral deposition has been approved for the treatment of ED. However, clinical studies showed that this route of administration is not effective in all patients. In addition, phentolamine and apomorphine are being evaluated as oral and sublingual therapies for ED, but neither compound has demonstrated efficacy across a broad range of subjects.

Potassium channel openers (KCO) and vasoactive intestinal polypeptide (VIP) also have been shown to be active via i.c. injection, but cost and stability issues could limit development of the latter. An alternative to the i.c. route is the use of glyceryl trinitrate (GTN) patches applied to the penis, which has been shown to be effective but produces side effects in both patient and partner.

As an alternative to pharmacological treatment, a variety of penile prostheses have been used to assist achievement of an erection. The short-term success rate is good, but problems with infection and ischemia, especially in diabetic men, make this type of treatment a final option rather than a first-line therapy.

For treatment of female low libido, flibanserin (marketed in the U.S. as Addyi® tablets) is taken as an oral pill and must be taken daily for effect, which is shown to be minimal compared to placebo. Side effects may include sleepiness and fainting as well as negative interactions with alcohol and other prescription medications commonly used by sexually active women.

Bremelanotide (marketed in the U.S. as Vyleesi® injection) is taken about 45 minutes before a sexual encounter and is self-administered as an injection in the thigh or abdomen. The best available evidence, which is limited, also shows minimal effect on the main outcome—number of satisfying sexual events—for women as compared to placebo. Nausea is a common side effect, which seems counter-productive.

Neither Addyi® tablets nor Vyleesi® injection target the female erection physiology of increased blood flow to the nipples and clitoris. Both medications are designed to increase sex drive. Vyleesi® injection activates melanocortin receptors. Addyi® tablets target serotonin receptors, but it is not well understood how these medications affect women's sexual desire.

One of the treatment options for BPH is medication that blocks alpha-1 receptors, such as terazosin, doxazosin, tamsulosin and alfuzosin. These drugs relax the bladder neck muscles and prostate muscles for easier urination. They can also improve the symptoms of BPH, such as frequency, urgency, nocturia, weak stream and incomplete emptying. However, they may also cause side effects such as low blood pressure, dizziness, headache, nasal congestion and retrograde ejaculation.

Accordingly, a need exists for improved and less invasive compositions and methods for treating sexual disorders in both males and females, and in treating BPH.

### SUMMARY OF THE INVENTION

The present invention is directed to a topical composition of magnesium sulfate or forskolin that can be applied topically to the epidermis of the penis, or by transdermal delivery to be absorbed through the connective tissue (equivalent to the dermis of typical skin) to provide relief of ED, premature ejaculation, low libido, or BPH in a male patient, or for use in treating orgasmic sexual dysfunction or low libido in a female patient. A topical composition of magnesium sulfate or forskolin can also be applied topically to genital areas to treat low libido in the female, by increasing female erections wherein there is increased blood flow to nipples and genitals and increased arousal.

Unlike the current treatment of ED with sildenafil citrate and other PDE 5 inhibitors, there are no systemic side effects or drug interactions when magnesium sulfate or forskolin is administered topically or by transdermal delivery. The composition is generally applied generously to the penis and massaged thoroughly. In females, the composition may be applied to the female genitals and massaged. In various embodiments, the composition may also be applied on a penis or sex toy or suppository inserted into the vagina or ano-rectal region, or as a condom lubricant, penile lubricant, vaginal lubricant or anal lubricant.

The erectile effect from topical or transdermal administration of magnesium sulfate or forskolin to the penis or female genitalia is localized, onset is within 2-5 minutes. This is unlike the PDE5 inhibitors used to treat erectile dysfunction in men, whose onset is 30 minutes to 1 hour. Duration of action is similar to the PDE5 inhibitors, and there are no drug interactions or contraindications. This is a very significant improvement.

In males, there is sustained erection with an ability to maintain an erection sufficient for satisfactory sexual performance and/or ejaculation. In females, there is increased arousal with increased female erections wherein there is increased blood flow to nipples and genitals. Female subjects have reported sleep orgasms without external stimulation.

The contact of the male penis with the female genital after topical magnesium sulfate or forskolin has been applied results in increased arousal of both the male and female and a more satisfactory sexual performance for both of them. Furthermore, there are increased blood levels of magnesium sulfate or forskolin from the topical or transdermal administration of magnesium sulfate or forskolin. For example, subjects have tasted magnesium in their mouth within one minute of topical administration. The increased blood levels of magnesium sulfate or forskolin from topical or transdermal administration results in increased testosterone with increased libido in both males and females lasting for several weeks. There are no side effects other than an allergy to the composition, and no drug interactions. No other prior medication can match such therapeutic effects.

Without being bound by theory, it is believed that the magnesium sulfate or forskolin is absorbed through the skin and connective tissue of the male penis, female genitalia and nipples as well as the mucosa of the vagina and ano-rectal region to relax the vascular smooth muscle tissue and improve blood flow to the erogenous zone or erectile tissue (e.g., the penis and female genitals and nipples). Within a few minutes of topical or transdermal application to the penis or female genitals, the subject begins to experience an increased sensation of heat in the penis, or female genitals, an evidence of increased blood flow, accompanied in males by an increase in penile size, length and turgidity. The increase in penile length, size and turgidity, significantly exceeds that obtained by topical or transdermal application of a control gel such as KY® jelly. Male subjects have the ability to achieve erections sufficient for sexual intercourse and the maintenance of erections after penetration. These effects reduce the symptoms of ED and significantly improve sexual performance and duration of performance. Both male and female subjects also have increased libido and quicker arousal for several weeks following application.

According to one aspect of the present invention, a composition for topical or transdermal administration of magnesium sulfate to a patient includes an effective amount of magnesium sulfate, or a pharmaceutically-acceptable salt thereof, and a pharmaceutically-acceptable carrier or excipient.

According to a second aspect of the present invention, a composition for topical or transdermal administration of forskolin to a patient includes an effective amount of forskolin (or a purified extract of Coleus Forskolii) or a pharmaceutically-acceptable salt thereof, and a pharmaceutically-acceptable carrier or excipient.

According to a third aspect of the present invention, a composition for topical or transdermal administration of magnesium sulfate, or a pharmaceutically-acceptable salt thereof, may be administered to a patient, the composition including an aqueous phase and a silicone phase provided as a water-in-silicone inverse phase emulsion. The aqueous phase includes magnesium sulfate in an effective amount, at least one water-soluble carrier, a humectant or emollient, at least one preservative, and water. The magnesium sulfate may be present in a concentration of about 10 to about 35 weight percent of the total weight of the composition. The at least one water-soluble carrier may be present in an amount of about 0.5 to about 6 weight percent of the total weight of the composition. The humectant or emollient may be selected from glycerin, sorbitol, sorbitan sesquioleate, aloe barbadensis leaf juice, propylene glycol, hyaluronic acid, propylheptyl caprylate, pentaclethra macroloba seed oil, butyrospermum parkii shea butter, carthamus tinctorius (safflower) seed oil, cocos nucifera oil, ceramide 3, caprylic/capric triglyceride or any combination thereof, and may be present in an amount of about 1 to about 10 weight percent of the total weight of the composition. The at least one preservative may be selected from phenoxyethanol, ethylhexylglycerin, caprylyl glycol, populus tremuloides bark extract, sodium levulinate, sodium anisate, sodium benzoate, potassium sorbate, lonicera japonica honeysuckle flower extract, lonicera caprifollium honeysuckle flower extract, leuconostoc/radish root ferment filtrate or any combination thereof, and may be present in an amount of about 0.5 to about 5 weight percent of the total weight of the composition. The water brings the composition to about 100 weight percent, and is preferably present in an amount of about 35 to about 65 weight percent of the total composition. These components are blended together to form the aqueous phase. The silicone phase includes a carrier oil, a water-insoluble carrier, a skin conditioning agent, a primary emulsifier, a texture enhancer, a base component, a dermal penetration enhancer, and a thickening agent. The carrier oil may be selected from jojoba oil, coconut oil, safflower seed oil, sunflower oil, castor oil, olive oil, squalane, meadowfoam seed oil, macadamia nut oil, argan oil, shea butter, vitamin E oil, aloe vera gel or almond oil, or any combination thereof, and may be present in an amount of about 0.1 to about 25 weight percent of the total weight of the composition. The water-insoluble carrier can include cyclomethicone, and may be present in an amount of about 5 to about 35 weight percent of the total weight of the composition. The skin conditioning agent may be selected from dimethicone, dimethiconol, or a combination thereof, and may be present in an amount of about 0.7 to about 2 weight percent of the total weight of the composition. The primary emulsifier may be selected from glyceryl stearate, tocopheryl acetate, tocopherol, squalane, stearic acid, pegylated forms of glyceryl stearate such as PEG-5 glyceryl stearate, PEG-100 stearate, PPG/PEG 18/18 dimethicone, cetyl alcohol, lecithin, hydrogenated lecithin, lanolin, squalene, or any combination thereof, and may be present in an amount of about 0.5 to about 5 weight percent of the total weight of the composition. The texture enhancer may be selected from C₁₂-C₁₅ alkyl benzoate, isopropyl myristate, isopropyl palmitate, ricinoleic acid, or any combination thereof, and may be present in an amount of about 0.5 to about 6 weight percent of the total weight of the composition. The aqueous phase and silicone phase are added together to provide the water-in-silicone inverse phase emulsion.

According to a fourth aspect of the present invention, a composition for topical or transdermal administration of forskolin, or a pharmaceutically-acceptable salt thereof, may be administered to a patient, the composition including an aqueous phase and a silicone phase provided as a water-in-silicone inverse phase emulsion. The aqueous phase includes forskolin, at least one water-soluble carrier, a pH adjuster, a humectant or emollient, at least one preservative, and water. The forskolin may be in the form of forskolin extract that is standardized to at least 10%-20% forskolin, such that forskolin is present in an amount of about 10 to about 35 weight percent of the total weight of the composition. The at least one water-soluble carrier may be selected from glycerine, propylene glycol, other polyhydric alcohols or mixtures thereof, and may be present in an amount of about 0.5 to about 6 weight percent of the total weight of the composition. The pH adjuster may be selected from sodium hydroxide or citric acid to adjust the pH of the composition to 5.5-7.0. The humectant or emollient may be selected from glycerin sorbitol, sorbitan sesquioleate, aloe barbadensis leaf juice, propylheptyl caprylate, pentaclethra macroloba seed oil, glucono delta lactone, or any combination thereof, and may be present in an amount of about 1 to about 10 weight percent of the total weight of the composition. The at least one preservative may be selected from phenoxyethanol, sodium benzoate, potassium sorbate, ethylhexylglycerin, caprylyl glycol, sodium levulinate, sodium anisate, sodium dehydro acetate, disodium EDTA, tetra sodium EDTA, butylated hydroxytoluene, parabens, or any combination thereof, and may be present in amount of about 0.5 to about 5 weight percent of the total weight of the composition. Water brings the composition to about 100 weight percent, and is preferably present in an amount of about 35 to about 65 weight percent of the total composition. The silicone phase includes a carrier oil, a water-insoluble carrier, a skin conditioning agent, a primary emulsifier, a texture enhancer, a base component, a dermal penetration enhancer, and a thickening agent. The carrier oil may be selected from jojoba oil, coconut oil, safflower seed oil, sunflower oil, castor oil, olive oil, squalane, meadowfoam seed oil, macadamia nut oil, argan oil, shea butter, vitamin E oil, aloe vera gel or almond oil, or any combination thereof, and may be present in an amount of about 0.1 to about 25 weight percent of the total weight of the composition. The water-insoluble carrier may include cyclomethicone, and may be present in an amount about 5 to about 35 weight percent of the total weight of the composition. The skin conditioning agent may be selected from dimethicone, dimethiconol, or any combination thereof, and may be present in an amount of about 0.7 to about 2 weight percent of the total weight of the composition. The primary emulsifier may be selected from cetearyl alcohol, PPG/PEG 18/18 dimethicone, sodium stearoyl glutamate, sodium lauryl sulfate, sodium laureth sulfate, diethanolamine, monoethanolamine, triethanolamine, or any combination thereof, and may be present in an amount of about 0.5 to about 5 weight percent of the total weight of the composition. The texture enhancer may be a C₁₂-C₁₅ alkyl benzoate, and may be present in an amount of about 0.5 to about 6 weight precent of the total weight of the composition. The base component may be selected from glycols such as propylene glycol or polyethylene glycol, an anhydrous silicone base, a lipophilic foam base, a transdermal cream base, or a mineral oil, or any combination thereof, and may be present in an amount of about 15 to about 80 weight percent of the total weight of the composition. The dermal penetration enhancer may be selected from volatile oils (e.g. essential oils such as such as lavender, peppermint, or eucalyptus), fixed oils (e.g. fatty acids) and polysaccharides, dimethyl sulfoxide, oleic acid, lauroglycol, isopropyl myristate, or polysorbates (Tween) such as polysorbate 20, 40, 60, 80, or any combination thereof, and may be present in an amount of about 0.1 to about 10 weight percent of the total weight of the composition. The thickening agent may be selected from xanthan gum, synthetic polymers, modified celluloses, cyclodextrin, or any combination thereof, and may be present at a concentration of about 0.5 to about 10 weight percent of the total weight of the composition. In various embodiments, the forskolin composition may be formulated as an oil-in-water emulsion, a water-in-oil emulsion, or a lipophilic foam.

According to a fifth aspect of the present invention, a composition for topical or transdermal administration of magnesium sulfate includes an aqueous phase and a silicone phase. The aqueous phase includes magnesium sulfate present in an amount of about 10 to about 35 weight percent of the total weight of the composition, at least one water-soluble carrier (such as glycerine, propylene glycol, other polyhydric alcohols, or any combination thereof) present in an amount of about 0.5 to about 6 weight percent of the total weight of the composition, a humectant or emollient (such as glycerin, propylheptyl caprylate, pentaclethra macroloba seed oil, glucono delta lactone, or any combination thereof) present in an amount of about 1 to about 10 weight percent of the total weight of the composition, at least one preservative (such as phenoxyethanol, ethylhexylglycerin, caprylyl glycol, sodium dehydro acetate, disodium EDTA, tetra sodium EDTA, butylated hydroxytoluene, or any combination thereof) present in an amount of about 0.5 to about 5.0 weight percent of the total weight of the composition, and water in an amount of about 35 to about 65 weight percent of the total weight of the composition. The silicone phase includes a water-insoluble carrier including cyclomethicone present in an amount of about 5 to about 35 weight percent of the total weight of the composition, a skin conditioning agent including dimethicone, dimethiconol, or any combination thereof, present in an amount of about 0.7 to about 2.0 weight percent of the total weight of the composition, a primary emulsifier (such as PPG/PEG 18/18 dimethicone, sodium stearoyl glutamate, or any combination thereof) present in an amount of about 0.5 to about 5.0 weight percent of the total weight of the composition, and a texture enhancer including C₁₂-C₁₅ alkyl benzoate present in an amount of about 0.5 to about 6.0 weight percent of the total weight of the composition. The dermal penetration enhancer may be selected from volatile oils (e.g. essential oils such as such as lavender, peppermint, or eucalyptus), fixed oils (e.g. fatty acids) and polysaccharides, dimethyl sulfoxide, oleic acid, lauroglycol, isopropyl myristate, or polysorbates (Tween) such as polysorbate 20, 40, 60, 80, or any combination thereof, and may be present in an amount of about 0.1 to about 10 weight percent of the total weight of the composition. The thickening agent may be selected from xanthan gum, synthetic polymers, modified celluloses, cyclodextrin, or any combination thereof, and may be present at a concentration of about 0.5 to about 10 weight percent of the total weight of the composition. In various embodiments, the forskolin composition may be formulated as an oil-in-water emulsion, a water-in-oil emulsion, or a lipophilic foam.

The aqueous and silicone phases are provided as a water-in-silicone inverse phase emulsion.

According to a sixth aspect of the present invention, a composition for topical or transdermal administration of forskolin includes forskolin present in an amount of about 10 to about 35 weight percent of the total weight of the composition, at least one water-soluble carrier (such as glycerine, propylene glycol, other polyhydric alcohols, or any combination thereof) present in an amount of about 0.5 to about 6 weight percent of the total weight of the composition, a pH adjuster (such as sodium hydroxide, citric acid, or any combination thereof) to adjust the pH to 5.5 to 7.0, a humectant or emollient (such as glycerin sorbitol, sorbitan sesquioleate, aloe barbadensis leaf juice, propylheptyl caprylate, pentaclethra macroloba seed oil, glucono delta lactone, or any combination thereof) present in an amount of about 1 to about 10 weight percent of the total weight of the composition, at least one preservative (such as phenoxyethanol, sodium benzoate, potassium sorbate, ethylhexylglycerin, caprylyl glycol, sodium levulinate, sodium anisate, sodium dehydro acetate, disodium EDTA, tetra sodium EDTA, butylated hydroxytoluene, parabens, or any combination thereof) present in an amount of about 0.5 to about 5.0 weight percent of the total weight of the composition, water present in an amount of about 35 to about 65 weight percent of the total weight of the composition, a carrier oil (such as jojoba oil, coconut oil, almond oil, or any combination thereof) present in an amount of about 0.1 to about 25 weight percent of the total weight of the composition, a water-insoluble carrier including cyclomethicone present in an amount of about 5 to about 35 weight percent of the total weight of the composition, a skin conditioning agent including dimethicone, dimethiconol, or any combination thereof, present in an amount of about 0.7 to about 2.0 weight percent of the total weight of the composition, a primary emulsifier (such as cetearyl alcohol, PPG/PEG 18/18 dimethicone, sodium stearoyl glutamate, sodium lauryl sulfate, sodium laureth sulfate, diethanolamine, monoethanolamine, triethanolamine, or any combination thereof) present in a concentration of about 0.5 to about 5.0 weight percent of the total weight of the composition, a texture enhancer including C₁₂-C₁₅ alkyl benzoate present in an amount of about 0.5 to about 6.0 weight percent of the total weight of the composition, a base component (such as propylene glycol and/or polyethylene glycol, lipophilic foams, water, anhydrous silicone base, lipophilic foam base, transdermal cream base, mineral oils, or any combination thereof), a dermal penetration enhancer (such as volatile oils including essential oils, fixed oils including fatty acids, polysaccharides, dimethyl sulfoxide, oleic acid, lauroglycol, isopropyl myristate, polysorbates (Tween) such as polysorbate 20, 40, 60, or 80, or any combination thereof) present in an amount of about 0.1 to about 10 weight percent of the total weight of the composition, and a thickening agent (such as xanthan gum, synthetic polymers, modified celluloses, cyclodextrin, or any combination thereof) present in an amount of about 0.5 to about 10 weight percent of the total weight of the composition.

According to a seventh aspect of the present invention, a method of making a magnesium sulfate composition includes blending an aqueous constituent in water to form an aqueous phase, wherein the aqueous constituent comprises magnesium sulfate in a concentration that ranges from about 20 to about 25 weight percent of the total weight of the composition, wherein the aqueous constituents are blended until the magnesium sulfate is dissolved in the aqueous phase; blending two or more water-insoluble constituents to form a silicone phase, wherein the two or more water-insoluble constituents comprise at least one water-insoluble carrier and a primary emulsifier; and mixing the aqueous phase into the silicone phase to provide a water-in-silicone inverse-phase emulsion.

According to an eighth aspect of the present invention, a method of making a forskolin composition includes providing forskolin (which may be pure forskolin in the form of forskolin powder or extract, such that forskolin is present in an amount of about 10 to about 35 weight percent of the total weight of the composition), and combining the forskolin with a water-soluble carrier, a pH adjuster, a humectant or emollient, at least one preservative, at least one carrier oil, water, a primary emulsifier, a water-insoluble carrier, a skin conditioning agent, a texture enhancer, a surfactant, and a base component.

According to a ninth aspect of the present invention, a method of treating ED, premature ejaculation, or low libido in a male patient includes administering a topical or transdermal formulation including magnesium sulfate, or a pharmaceutically-acceptable salt thereof, to an erogenous and/or ano-rectal zone of the male patient.

According to a tenth aspect of the present invention, a method of treating ED, premature ejaculation, or low libido in a male patient includes administering a topical or transdermal formulation including forskolin, or a pharmaceutically-acceptable salt thereof, to an erogenous and/or ano-rectal zone of the male patient.

According to a eleventh aspect of the present invention, a method of increasing the size and/or turgidity of an erectile tissue in a male patient includes administering a topical or transdermal formulation including magnesium sulfate, or a pharmaceutically-acceptable salt thereof, to the erectile tissue of the male patient.

According to a twelfth aspect of the present invention, a method of increasing the size and/or turgidity of an erectile tissue in a male patient includes administering a topical or transdermal formulation including forskolin, or a pharmaceutically-acceptable salt thereof, to the erectile tissue of the male patient.

According to a thirteenth aspect of the present invention, a method of treating orgasmic sexual dysfunction or low libido in a female patient includes administering a topical or transdermal formulation including magnesium sulfate, or a pharmaceutically-acceptable salt thereof, to an erogenous and/or ano-rectal zone of the female patient.

According to a fourteenth aspect of the present invention, a method of treating orgasmic sexual dysfunction or low libido in a female patient includes administering a topical or transdermal formulation including forskolin, or a pharmaceutically-acceptable salt thereof, to an erogenous and/or ano-rectal zone of the female patient.

According to a fifteenth aspect of the present invention, a method of increasing the size, sensitivity and arousability of an erectile tissue in a female patient includes administering a topical or transdermal formulation including magnesium sulfate, or a pharmaceutically-acceptable salt thereof, to the erectile tissue of the female patient.

According to a sixteenth aspect of the present invention, a method of increasing the size, sensitivity and arousability of an erectile tissue in a female patient includes administering a topical or transdermal formulation including forskolin, or a pharmaceutically-acceptable salt thereof, to the erectile tissue of the female patient.

According to a seventeenth aspect of the present invention, a method of treating orgasmic sexual dysfunction or low libido in a female patient includes administering a topical or transdermal formulation including magnesium sulfate, or a pharmaceutically-acceptable salt thereof, to an erogenous and/or ano-rectal zone of the female patient to increase the size, sensitivity and arousability of an erectile tissue in the female patient, resulting in increased sexual arousal.

According to an eighteenth aspect of the present invention, a method of treating orgasmic sexual dysfunction or low libido in a female patient includes administering a topical or transdermal formulation including forskolin, or a pharmaceutically-acceptable salt thereof, to an erogenous and/or ano-rectal zone of the female patient to increase the size, sensitivity and arousability of an erectile tissue in the female patient, resulting in increased sexual arousal.

According to a nineteenth aspect of the present invention, a method of treating benign prostatic hyperplasia (BPH) in a male patient includes administering a topical or transdermal formulation including magnesium sulfate, or a pharmaceutically-acceptable salt thereof, to the male patient's penis, ano-rectal or prostate region.

According to a twentieth aspect of the present invention, a method of treating BPH in a male patient includes administering a topical or transdermal formulation including forskolin, or a pharmaceutically-acceptable salt thereof, to the male patient's penis, ano-rectal or prostate region.

### DETAILED DESCRIPTION

The present invention provides a method of using magnesium sulfate or forskolin to treat both organic, psychogenic and mixed ED, or BPH in the male and to treat low libido/orgasmic sexual dysfunction (female sexual interest/arousal disorder) in the female.

Furthermore, the present invention provides a novel medicament useful for the treatment of ED, premature ejaculation, low libido, and BPH in the male and decreased libido/orgasmic sexual dysfunction in the female. Such disorders can be treated by the non-invasive topical application or transdermal delivery on the penis, scrotum, nipples, or ano-rectal region of a male, or the breasts, nipples, ano-rectal region, or genital area (vulva, clitoris and vagina) in the female, of a formulation described herein. It may also be applied on a penis, finger, sex toy, condom lubricant, penile lubricant, vaginal lubricant, anal lubricant or suppository before the penis, finger, sex toy, condom lubricant, or suppository is inserted into the vagina or ano-rectal region.

### Definitions

As used herein, the recited terms have the following meanings. All other terms and phrases used in this specification have their ordinary meanings as one of ordinary skill in the art would understand. Such ordinary meanings may be obtained by reference to technical dictionaries, such as Hawley's Condensed Chemical Dictionary 14.sup.th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more ingredients can refer to one; one or two; one, two, or three; one two, three, or four; five, six, seven, eight, nine, or about ten ingredients.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than are cited integer at each end of the range. Unless indicated otherwise herein, the term “about” is intended to include values, e.g., weight percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in the irrespective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into subranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

“Penetration enhancement” or “permeation enhancement” refers to an increase in the permeability of the skin or mucosal tissue (e.g., vaginal mucosa or anal mucosa) to a pharmacologically active agent in the presence of a penetration enhancer, i.e., so that the rate at which the agent permeates the skin (i.e., the “flux” of the agent through the body surface) is increased relative to the rate that would be obtained in the absence of penetration enhancer. The enhanced permeation effected through the use of such enhancers can be observed by measuring the rate of diffusion of an active through animal or human skin using, for example a Franz diffusion apparatus, as known in the art.

The terms “treating,” “treat,” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes medical, therapeutic, and/or prophylactic administration, as appropriate.

“Patient” refers to a human or non-human animal, either male or female.

“Topical” or “transdermal” administration includes internal or external administration to the genitals (e.g., ano-rectal region and/or vagina), and includes transmucosal administration, including intra-vaginal and intra-anal administration.

“Erogenous zone” includes the genital region (interior and exterior), ano-rectal region (interior and exterior), and breast/nipples.

“Size and/or turgidity” refers to the length or diameter and/or firmness or rigidity of an erectile tissue in a male patient.

“Erectile tissue” refers to penile, nipple, or clitoral tissue.

“Orgasmic sexual dysfunction” refers to reduced female sexual interest/arousal disorder, including low interest in sex, with deficient or absent sexual fantasies and desire for sexual activity, and/or inability to achieve orgasm from sexual contact.

“Enhanced penile erection” refers to an erection of increased size and/or turgidity.

“Penetration” refers to insertion into the vagina or ano-rectal contact.

“Improved urinary function” refers to better ability to urinate/void the bladder, and/or a reduction in urinary tract infections (UTIs).

### Therapeutic Formulations

### Active Agents—Magnesium Sulfate and Forskolin

The active agents of the present invention are envisioned for the treatment of ED in humans and animals, such as male ED, and for the treatment of BPH, as well as to treat female sexual dysfunction, including low libido and orgasmic sexual dysfunction related to clitoral disturbances.

### Magnesium Sulfate

Magnesium sulfate is a chemical compound having the chemical formula MgSO⁴, consisting of magnesium cations Mg⁺² and sulfate anions SO₂⁻⁴. It is a white crystalline solid, soluble in water, but not in ethanol. In various embodiments, magnesium sulfate is in the form of magnesium sulfate heptahydrate.

Magnesium sulfate increases smooth muscle relaxation, reduces striated muscle contractions and blocks peripheral neuromuscular transmission by reducing acetylcholine release at the myoneural junction. Additionally, magnesium sulfate inhibits Ca²⁺ influx through dihydropyridine-sensitive, voltage-dependent channels. Magnesium sulfate activates adenylate cyclase, via receptor associated G proteins to increase cyclic 3′,5′ adenosine monophosphate formation (cAMP). Increased cAMP synthesis results in activation of cAMP-dependent protein kinase A (PKA), and PKA-mediated smooth muscle relaxation. PKA-mediated vascular smooth muscle relaxation includes decreases of intracellular calcium concentrations, and decreases in calcium sensitivity by myosin light chain kinase (MLCK) and inhibition of RhoA/Rho kinase (ROCK).

MLCK plays a central role in the initiation of smooth muscle contraction and many nonmuscle motile processes owing to its Ca²⁺/calmodulin-dependent phosphorylation of myosin regulatory light chain (RLC). The sensitivity of MLCK to activation by Ca²⁺/calmodulin is diminished upon phosphorylation at a regulatory site A by calmodulin kinase II, resulting in desensitization of the contractile elements to elevated levels of intracellular Ca²⁺.

Phosphorylation of MLCK has been shown to desensitize light chain phosphorylation to Ca²⁺. Initial investigations on the phosphorylation-dependent desensitization of MLCK focused on the possibility that phosphorylation of MLCK by cAMP is a mechanism whereby increases in cAMP and activation of PKA lead to the relaxation of smooth muscle. ROCK is a key regulator of smooth muscle contractility. RhoA is a small GTPase that binds to several surface receptors that activate ROCK when it is bound to guanosine triphosphate (GTP). Inhibition of ROCK results in smooth muscle relaxation. In addition to PKA, effects of cAMP can be mediated independently of PKA by exchange proteins directly activated by cAMP (Epac). Therefore, magnesium sulfate induced increase in cAMP production within smooth muscle cells, leads to relaxation of vascular smooth muscle resulting in increased blood flow to the sex organs, within minutes, in both human and non-human, male and females.

Magnesium sulfate by its actions will also result in relaxation of the smooth muscle of the prostate and help relieve symptoms of BPH. Prostate smooth muscle contraction and prostate growth drive urethral obstruction in BPH, resulting in impairments of voiding and bladder emptying, and finally lower urinary tract symptoms (LUTS).

It has been shown that magnesium sulfate is a potent vascular smooth muscle relaxant. Thus, magnesium sulfate is of interest for use in therapy, specifically for the treatment of a variety of conditions including ED, premature ejaculation, low libido, and BPH in the male and to treat low libido in the female.

The biochemical, physiological, and clinical effects of magnesium sulfate suggest its utility in a variety of disease states such as ED, premature ejaculation, low libido, and BPH in males, where modulation/relaxation of smooth muscle and increased blood flow to the penis is desirable. A topical composition of magnesium sulfate can also be applied topically to the genital area (vulva, vagina and clitoris) to treat low libido (female sexual interest/arousal disorder) in the female, by increasing female erections wherein there is increased blood flow to their nipples and their clitoris and increased arousal.

Increasing smooth muscle relaxation of the corpus is an important step in the physiology of penile erections. A penile erection is caused by neural stimuli that ultimately cause vasodilation of the arteries and sinusoidal spaces of the corpus cavernosum. Research indicates that smooth muscle relaxation of the corpus plays a central role in this vasodilation.

The present invention provides a safe and effective method of treating ED, premature ejaculation, low libido, and BPH in the male and low libido/orgasmic sexual dysfunction in the female, without the need for oral drugs, injectable drugs or invasive treatments. Magnesium sulfate is a natural mineral and when applied topically or by transdermal delivery has few known side effects or drug interactions. Additionally, the method can be used in conjunction with any other treatments for ED, including lifestyle changes and psychological therapy.

### Forskolin

Forskolin (7beta-acetoxy-8, 13-epoxy-1a, 6β, 9a-trihydroxy-labd-14-en-11-one) is a labdane diterpene derived from the roots of the Indian Coleus forskohlii plant (Plectranthus barbatus), a plant in the mint family. It is a lipid-soluble compound that can penetrate cell membranes and stimulates the enzyme adenylate cyclase. In the eye, adenylate cyclase stimulates ciliary epithelium to activate cyclic adenosine monophosphate, which decreases intraocular pressure (TOP) by reducing aqueous humor inflow. The topical application of forskolin is capable of reducing TOP in animals (Caprioli and Sears, 1983, 1984; Caprioli et al., 1984) and humans (Burstein et al., 1984). In some embodiments, the forskolin is in the form of a purified extract of Coleus forskohlii.

Forskolin is water soluble and in some embodiments, the forskolin is extracted from the Coleus Forskohlii plant utilizing solvents such as betahydroxycyclodextrine, hexane, ethyl acetate, dichloromethane, isopropyl alcohol, cyclohexanol, methanol, ethanol and butanol to obtain at least a technical grade of purity of ≥70%-99% pure forskolin. In other embodiments, the forskolin is in a crude state that has not been fully refined when extracted from the Coleus Forskohlii plant and can contain components in addition to forskolin having a purity of ≥40%-65% pure forskolin.

Elevation of intracellular cAMP after activation of G coupled receptors by vasorelaxing hormones such as adrenaline, noradrenaline and the endothelium-derived prostaglandin I2 (PGI2) induces a rapid and efficient relaxation of mature differentiated smooth muscle cells (SMCs). The cAMP elevating agent forskolin induces a relaxant effect in vascular smooth muscle cells (VSMCs) in vivo which is potentiated by inhibitors of phosphodiesterase type 3 (PDE3) and of phosphodiesterase type 4 (PDE4), the two main PDE isoforms expressed in VSMCs. In SMCs, cAMP contributes to muscle relaxation through two different mechanisms; one through the stimulation of the calcium pump at the sarcolemmal membrane (Ca extrusion) and sarcoplasmic reticulum (Ca accumulation), and the other through the de-phosphorylation of MLCK. De-phosphorylation of MLCK is accomplished by the myosin light chain phosphatase (MLCP) which is well known to be activated upon phosphorylation by the cAMP target PKA or the cGMP dependent protein kinase G (PKG). Conversely, when phosphorylated by ROCK or protein kinase C (PKC), MLCP activity is inhibited, resulting in contraction.

Forskolin treats ED, premature ejaculation and low libido in the male, by producing smooth muscle relaxation in the corpus cavernosum, allowing inflow of blood. There is increased blood flow into the expanding sinusoids of the corpora cavernosa. Forskolin absorption results in improved blood flow to the patient's prostate region, increased testosterone level in the patient, improved urinary function in the patient, or any combination thereof. In females, forskolin absorption increases blood flow to the erectile tissues of the genitalia as well as the nipples. This treats orgasmic sexual dysfunction and low libido by increasing size, sensitivity and arousability of the female genitalia and nipples.

### Sexual Disorders

According to one or more embodiments, the compositions and formulations described herein are useful in the treatment of various sexual disorders, including but not limited to ED, premature ejaculation, or low libido in a male patient, and orgasmic sexual dysfunction or low libido in a female patient. In various embodiments, the compositions are administered as a topical or transdermal formulation to an erogenous zone of a male or female patient. In some embodiments, the compositions may be used to increase the size and/or turgidity of an erectile tissue in a male patient. In certain embodiments, the compositions may be used to increase the size, sensitivity and arousability of an erectile tissue in a female patient by administering the compositions to the erectile tissue of the female patient, which can in some embodiments, result in increased sexual arousal.

In various embodiments, relaxing the vascular smooth muscle tissue includes inhibiting Ca++ influx into the vascular smooth muscle tissue. In some embodiments, relaxing the vascular smooth muscle tissue includes activating an adenylate cyclase enzyme in the vascular smooth muscle tissue. In certain embodiments, relaxing the vascular smooth muscle tissue includes increasing formation of cAMP in the vascular smooth muscle tissue. In several embodiments, relaxing the vascular smooth muscle tissue includes activating a cAMP-dependent protein kinase in the vascular smooth muscle tissue.

In certain embodiments, forskolin, magnesium sulfate, or a plurality of Mg++ ions may be absorbed through the male or female patient's skin and connective tissue in the erogenous zone or erectile tissue. In several embodiments, the forskolin, magnesium sulfate, or the plurality of Mg++ ions are absorbed and generate a physiological effect in less than 10 minutes, for example in 2 to 5 minutes.

In one or more embodiments, the physiological effect includes improved blood flow to the erogenous zone or erectile tissue. In other embodiments, the physiological effect includes an increased testosterone level in the male or female patient. In various embodiments, the physiological effect includes an increased libido in the male or female patient. In several embodiments, the physiological effect includes an increased sexual arousal in the male or female patient.

In certain embodiments, the patient is a male patient, the physiological effect may be a penile erection, and the penile erection may be an enhanced penile erection. In these embodiments, the physiological effect is enhanced maintenance of the penile erection or enhanced penile erection after penetration. The formulation may be applied to the male patient's penis, scrotum, nipples, exterior or interior surface of the male patient's ano-rectal region, or a finger or sex toy.

In various embodiments, the patient is a female patient, the physiological effect may be a reduction in orgasmic sexual dysfunction, and the formulation may be applied to one or more of the female patient's breasts. The formulation may also be applied to the female patient's nipples, ano-rectal region, an exterior or interior surface of the female patient's genitals, an interior surface of the female patient's ano-rectal region or vagina, or to a sex toy, a finger, a penis, or a condom lubricant, penile lubricant, vaginal lubricant, or anal lubricant.

### Benign Prostatic Hyperplasia (BPH)

According to one or more embodiments, the compositions and formulations described herein are useful in the treatment of BPH in a male patient by administering the compositions and formulations to the male patient's penis, ano-rectal region, or prostrate region. In some embodiments, topical or transdermal application of the compositions and formulations reduces the size of the patient's prostate gland, or reduces an enlargement rate of the patient's prostate gland.

In one or more embodiments, the methods further include relaxing a vascular smooth muscle tissue in the patient's prostrate region. Relaxing the vascular smooth muscle tissue can include inhibiting Ca++ influx into the vascular smooth muscle tissue, activating an adenylate cyclase enzyme in the vascular smooth muscle tissue, increasing formation of cyclic 3′,5′ adenosine monophosphate (cAMP) in the vascular smooth muscle tissue, activating a cAMP-dependent protein kinase (PKA) in the vascular smooth muscle tissue, or any combination thereof.

In various embodiments, forskolin, magnesium sulfate or a plurality of Mg++ ions are absorbed through the male patient's skin and connective tissue in the prostate region. This absorption can result in improved blood flow to the patient's prostate region, increased testosterone level in the patient, improved urinary function in the patient, or any combination thereof.

### Dosing and Administration Procedures

The compositions and formulations described herein typically contain at least 0.1 weight precent of an active agent (e.g., magnesium sulfate or forskolin). The percentage of active(s) in the compositions and formulations can vary and may conveniently be about 0.2 weight percent to about 50 weight percent, about 0.5 weight percent to about 40 weight percent, about 1 weight percent to about 30 weight percent, about 2 weight percent to about 25 weight percent, or about 3 weight percent to about 20 weight percent, of the weight of a given unit dosage form. The amount of active in such therapeutically useful compositions is such that an effective dosage level can be attained. In one embodiment, the active is present in an amount of about 12.0 to about 25.0 weight percent based on the total weight of the composition. In another embodiment, the active is present in an amount of about 0.1 to about 1.0 weight percent based on the total weight of the composition.

With respect to treating sexual dysfunction and particularly ED in human or animal males and low libido/orgasmic sexual dysfunction in human or animal females, topical or transdermal administration of the compounds of the invention is the preferred route. Topical or transdermal administration in the male is the most convenient and avoids the disadvantages associated with intracavernosal and oral administration. For male or female patients suffering from a swallowing disorder or from impairment of drug absorption after oral administration, the topical or transdermal administration of the drug confers a significant advantage.

For veterinary use, a compound of magnesium sulfate or a nontoxic salt thereof, is administered as a suitably acceptable formulation in accordance with normal veterinary practice. The veterinarian can readily determine the dosing regimen for the topical or transdermal route of administration that is most appropriate for a particular male or female animal.

In a particular embodiment, the present invention includes a pharmaceutical composition for the curative or prophylactic treatment of ED in a male animal, including man, including a magnesium sulfate or forskolin or a pharmaceutically acceptable salt thereof, together with a pharmaceutically acceptable diluent or carrier. A topical composition of magnesium sulfate or forskolin can also be applied topically or transdermally to human and non human female genitals (vulva, vagina and clitoris), either directly, or applied on a penis, condom lubricant, penile lubricant, vaginal lubricant, anal lubricant, sex toy or suppository inserted into the vagina or ano-rectal region, to treat low libido/orgasmic sexual dysfunction in the female, by increasing female erections wherein there is increased blood flow to their nipples and their clitoris and increased arousal.

The following examples are intended to illustrate the above invention and should not be construed as to narrow its scope. One skilled in the art will readily recognize that the examples suggest many other ways in which the invention could be practiced. It should be understood that numerous variations and modifications may be made while remaining within the scope of the invention.

### Enhanced Delivery Techniques

Transdermal delivery can be carried out by methods known in the art or as described herein, including, for example, methods directed to 1) the use of chemical penetration enhancers or skin penetration enhancers such as dimethyl sulfoxide, oleic acid, lauroglycol, isopropyl myristate, cetostearyl alcohol, or polysorbates (Tween) such as polysorbate 20, 40, 60, or 80; 2) liposome-mediated delivery; 3) iontophoresis, also known as transdermal electromotive drug administration; 4) electroporation; 5) sonophoresis (ultrasound waves); 6) mechanical (e.g., microporation) devices, and/or 7) air pressure. Methods suitable for transdermal delivery of the agents described herein can include, for example, methods directed to enhancing the transport of material across the skin pores by increasing the rate of transport across existing pores or by amplifying the number of available skin pores through the creation of artificial pores.

Transdermal delivery can be carried out by the use of chemical or penetration enhancers, including for example, a pharmaceutically acceptable oil of vegetable, nut, synthetic or animal origin including emu oil, ethoxylated oil, polyethylene glycol (PEG), linoleic acid, ethanol, methanol, and/or agents which delipidize the stratum corneum. Suitable oils include meadow foam oil, castor oil, jojoba oil, corn oil, sunflower oil, sesame oil, and emu oil, all of which may be optionally ethoxylated. Examples include those oils and components described in U.S. Pat. No. 7,291,591 (Fishman); U.S. Pat. No. 7,052,715 (Fishman); U.S. Pat. No. 7,033,998 (Fishman); U.S. Pat. No. 6,951,658 (Pearson et al.); U.S. Pat. No. 6,946,144 (Jordan); U.S. Pat. No. 6,759,056 (Jordan); U.S. Pat. No. 6,750,291 (Kim et al.); U.S. Pat. No.6,720,001 (Chen et al.); U.S. Pat. No. 6,224,853 (Steel et al.); and U.S. Pat. No. 5,695,779 (Mori).

In addition, transdermal patches can be used for the topical or transdermal delivery of a composition described herein. A patch can also be adapted for delivery of dry or lyophilized forms of the compositions described herein, for example, using techniques as described in U.S. Pat. No. 5,983,135 (Avrahami). Other patch technology that can be employed in conjunction with the compositions described herein, e.g., in a reservoir of the patch, include the patch technology described in U.S. Pat. No. 6,835,392 (Hsu et al.).

Transdermal delivery can also be carried out by liposome mediated delivery methods (e.g., delivery facilitated by application of lipophilic membrane compositions). Suitable examples may include those described in U.S. Pat. No. 5,910,306 (Alving et al.); U.S. Pat. No. 5,718,914 (Foldvari); and U.S. Pat. No. 5,064,655 (Uster et al.). Transdermal delivery systems can also be employed in conjunction with a wide variety of iontophoresis or electrotransport systems. Illustrative electrotransport drug delivery systems that can be used in conjunction with the topical formulations herein are described by U.S. Pat. No. 5,147,296 (Theeuwes et al.); U.S. Pat. No. 5,169,382 (Theeuwes et al.); and U.S. Pat. No. 5,169,383 (Gyory et al.).

When a sonophoresis technique is used, one ultrasonic frequency can be applied to the skin, or two or more different ultrasonic frequencies can be applied to the skin (e.g., one low and one high ultrasonic frequency). As with the other techniques described above, this technique can be used in combination with other techniques, such as prior to the topical or transdermal application of a composition described herein, including the application of a transdermal patch. Iontophoresis or electroporation techniques that can be employed are further described in U.S. Patent Publication No. 2004/0210184 (Kost et al.).

Another transdermal drug delivery technique that can be used in conjunction with the compositions described herein includes employing a device to use air pressure to inject a small stream of the composition through the top layers of the skin without the aid of a needle. The air pressure gun can be the same as or similar to the devise used to provide vaccines to children. Small, disposable pen-like devices are also suitable, as for diabetics who take insulin daily.

### Pharmaceutical Carriers

The following are examples of therapeutic compositions that can be used for the effective treatment of ED, premature ejaculation and low libido. In the various therapeutic compositions described above and in the examples below, the specific active agent is magnesium sulfate or forskolin.

A topical or transdermal composition typically includes an active agent and a pharmaceutically acceptable carrier for topical or transdermal administration. Active agents can be combined with a pharmaceutically acceptable carrier or diluent to produce a pharmaceutical composition or formulation. In such pharmaceutical formulations, the active agents or therapeutic composition can be combined with a carrier that is physiologically compatible with the skin or mucosal tissue (e.g., vaginal mucosa or anal mucosa) of a human or animal to which it is topically administered or by transdermal delivery. Typically, the carrier is substantially inactive, with the exception of its intrinsic surfactant properties which may aid in the production of a solution or suspension of the active ingredients. The compositions may include other physiologically active constituents that do not interfere with the efficacy of the active agents in the composition. In some embodiments, the carriers can be liquid or gel-based materials for use in liquid or gel formulations. The specific formulations depend, in part, upon the desired routes or modes of administration.

Suitable carrier materials include any carrier or vehicle commonly used as a base for solutions, liquids, dispersions, emulsions, solids, gels, creams, ointment, lotions, pastes, foams or aerosol spray, for topical or transdermal administration. Examples include emulsifying agents, inert carriers including hydrocarbon bases, emulsifying bases, non-toxic solvents or water-soluble bases. In some embodiments, the liquid is formulated as a liniment, body rub, serum, or sprayable solution. In one or more embodiments, the liquid is formulated as a sprayable solution or serum having a viscosity that ranges from about 15 to about 4,500 cps at 25 degrees Celsius. In certain embodiments, the liquid is formulated as a lotion or a liniment having a viscosity that ranges from about 3,000 to about 30,000 cps at 25 degrees Celsius. In various embodiments, the solid is formulated as a stick-type solid. In several embodiments, the composition is formulated as a cream, gel, paste, or ointment having a viscosity that ranges from 15,000 to about 250,000 cps at 25 degrees Celsius.

Suitable carrier materials include any carrier or vehicle commonly used as a base for solutions, dispersions, emulsions, gels, creams, ointment, lotions, pastes, foams or aerosol spray, for topical or transdermal administration. Examples include emulsifying agents, inert carriers including hydrocarbon bases, emulsifying bases, non-toxic solvents or water-soluble bases.

Many suitable liquid or gel-based carriers are well-known in the art. The carrier should be able to dissolve or disperse an active at an effective level, optionally with the aid of non-toxic surfactants. Examples include water, physiological salt solutions, alcohols (e.g., methanol, ethanol, propanol, or butanol), glycerol, glycols (e.g., ethylene glycol, propylene glycol, or ethoxy diglycol), polyethyleneglycol (e.g., MW 400 to 20,000), water-alcohol/glycol blends, and the like. Suitable carriers and diluents for certain embodiments include, for example, water, saline, isotonic saline solutions, for example, phosphate-buffered saline, aqueous dextrose, glycerol, ethoxy diglycol, dimethyl sulfoxide (DMSO), and the like, or combinations thereof.

Suitable carriers further include aqueous and oleaginous carriers such as, for example, white petrolatum, isopropyl myristate, lanolin or lanolin alcohols, mineral oil, fragrant or essential oil, nasturtium extract oil, sorbitan mono-oleate, cetostearyl alcohol (together or in various combinations),and detergents (e.g., polysorbates (Tweens) such as polysorbate 20, 40, 60, or 80; polyoxyl stearate; or sodium lauryl sulfate). One or more carrier materials can be mixed with water to form a lotion, gel, cream, foam or aerosol spray, semi-solid composition, or the like. Other suitable carriers include water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether, water, or combinations thereof. For example, emulsions containing water, glycerol stearate, glycerin, mineral oil, synthetic spermaceti, cetyl alcohol, or combinations thereof, may beused. Preservatives may also be included in the carrier, such as one or more of butylparaben, methylparaben, propylparaben, benzyl alcohol, ethylene diamine tetra acetate salts, phenoxyethanol, ethylhexylglycerin, caprylyl glycol, populus tremuloides bark extract, sodium levulinate, sodium anisate, sodium benzoate, potassium sorbate, lonicera japonica honeysuckle flower extract, lonicera caprifollium honeysuckle flower extract, and/or Leuconostoc/radish root ferment filtrate. The composition of the carrier can be varied so long as it does not interfere significantly with the pharmacological activity of the active ingredients of the therapeutic composition.

In some embodiments, a water-soluble carrier is used. The water-soluble carrier may be selected from glycerine, ethyhexyl glycerine, propylene glycol, dipropylene glycol, butylene glycol, ethoxydiglycol, a polyhydric alcohol, or any combination thereof.

In certain embodiments, a water-insoluble carrier is used. The water-insoluble carrier can include a cyclomethicone. In one embodiment, the cyclomethicone includes cyclodimethylpentasiloxane. In various embodiments, the cyclodimethylpentasiloxane is present in an amount of about 5.0 to about 25.0 weight percent based on the total weight of the composition.

In any of the therapeutic compositions of the present invention, the carrier, when present, can be a suitable topical carrier such as mineral oil, ethylene glycol, propylene glycol, polyethylene glycol, or a combination thereof. In any embodiment, the pharmaceutically acceptable carrier can include one or more oils, including emu oil. In one embodiment, the composition includes 1.0 weight percent to 10 weight percent, 2 weight percent to 15 weight percent, or 5 weight percent to 10 weight percent, of one or more non-emu oil carriers. In one or more embodiments, the composition includes an oil concentration that is less than 10 weight percent based on the total weight of the composition. In some embodiments, the composition is oil free.

The administration can be the topical or transdermal application of a gel, a jelly, a cream, a lotion, a wax, an ointment, a solution, a paste, an aerosol, a patch, and/or a combination thereof. Suitable pharmaceutically acceptable carriers include, but are not limited to, creams such as Cetaphil® Moisturising Cream (Galderma Laboratories, L.P.), QV Cream (Lision Hong), sorbolene, or the like. In some embodiments, the pharmaceutical acceptable carrier includes a lotion, such as Alpha Keri Moisturizing Lotion (Mentholatum), DermaVeen Moisturizing Lotion (DermaTech Laboratories), QVSkin Lotion (Lision Hong), Cetaphil® Moisturizing Lotion (Galderma Laboratories, L.P.), or the like.

In one embodiment, the carrier can be a pluronic lecithin organogel (PLO) gel. A PLO gel contains isopropyl palmitate (a non-oleaginous emollient), soy lecithin (mixture of phospholipids),water, and Pluronic® F-127 surfactant.

A fragrance can be included in any suitable amount (e.g., 0.01% by weight to about 3% by weight) or the fragrance can be omitted from the formulation. The composition can be applied to the skin in any suitable dosing regimen, for example, as recommended by a clinician or medical practitioner. One advantageous dosing schedule can involve applying the compositions as needed prior to sexual intercourse.

### Nanoparticles

In one embodiment, the composition includes nanoparticles that may be composed of materials such as polymers, lipids (e.g., liposomes) or dendrimers, such as polyamidoamine (PAMAM), polyethylenimine (PEI), polypropyleneimine (PPI), or polylysine containing dendrimers. For example, the use of nanoemulsion containing nanoparticles including magnesium sulfate provides for different physical and chemical properties, for instance, nanoparticles may allow for deeper penetration into the skin, delivering treatment to more layers of skin and beyond. In one embodiment, the nanoparticles are coated with and/or embedded with (encapsulate) the composition. The nanoparticles may be formed using techniques such as electrospraying. In one embodiment, the nanoparticles may be formed by grinding particles of magnesium sulfate to a desired size. In one embodiment, the nanoparticles may include a surface coating.

In one embodiment, the nanoparticles may be from about 1 nm to about 100 nm, about 100 nm to about 2,500 nm, or about 2,500 nm to about 10,000 nm in diameter. In one embodiment, nanoparticles may be formed of polymers or surfactants.

Polymers and surfactants include but are not limited to non-ionic surfactants, anionic surfactants, e.g., carboxylates such as alkyl carboxylates-fatty acid salts or carboxylate fluoro surfactants; sulfates such as alkyl sulfates (RCOO) (e.g. sodium laurylsulfate) or alkyl ether sulfates (e.g., sodium laureth sulfate); sulfonates, e.g., docusates (e.g., dioctylsodium sulfosuccinate) or alkyl benzene sulfonates; and phosphate esters such as alkyl aryl etherphosphates or alkyl ether phosphates; cationic surfactants including fatty amine salts and quaternary ammoniums; zitterionic surfactants such as those with a quaternary amine group and a sulfonic or carboxyl group, natural polymers and synthetic polymers including but not limited to alginate, hyaluronate, chitosan, gelatin, cellulose, e.g., ethylcellulose, polyvinyl pyrrolidone (PVP), polyethylene glycol (PEG) or acylates. In one embodiment, the surfactant includes polyoxyethylene, poloxamer, poloxamine, or polysorbate.

In one embodiment, the nanoparticles are formed of synthetic polymers including but not limited to poly-lactide, e.g., methoxyPEG-polylactide, polyglycolide, polyacrylates, e.g., poly(alkylcyanoacrylate), or polycapronates. In one embodiment, the nanoparticles are formed of natural polymers including but not limited to albumin, gelatin, alginate, collagen, chitosan, dextran, orelastin.

In one embodiment, the nanoparticles comprise degradable (aliphatic polyesters), and nondegradable (polyacrylates) polymers.

In one embodiment, the nanoparticles comprise synthetic polymers including but not limited topoly(DL-lactide co-glycolide) (PLGA), polycaprolactone (PCL), poly (lactic acid) (PLA), polyalkylcyanoacrylate, polyacrylic acid (PAA) and poly (methyl methacrylates).

In one embodiment, the nanoparticles do not include vinyl pyrrolidone, vinyl acetate or dioctylsodium sulfosuccinate.

In one embodiment, the nanoparticles have a surface modification, for example, a surface modified with PEG, polyvinyl alcohol (PVA), PEI, PVP, gold (Au), silica (SiO₂), chitosan or dextran.

### Gelling Agents and Thickening Agents

The compositions described herein can include one or more gelling agents to increase the viscosity of the composition. The gelling agent can be replaced with a thickening agent, or a thickening agent can be added in addition to the gelling agent.

Examples of gelling agents and thickening agents, include, but are not limited to, fatty acids, fatty acid salts and esters, fatty alcohols, synthetic polymers, modified celluloses, xanthan gum, or combinations thereof. Examples of suitable synthetic polymers include PEG, PVP, PVA, various Pluronics (poloxamers), or carbomers (e.g., Carbomer 940 or Carbomer 934). Examples of suitable modified celluloses include methyl cellulose, carboxymethylcellulose (CMC), hydroxyethylcellulose (HEC), hydroxymethyl cellulose (HMC), hydroxypropyl cellulose (HPC), hydroxypropyl-methylcellulose (HPMC), or other cellulose-based gelling agents.

A variety of gelling agents are commercially available and can be obtained in many suitable molecular weights and ranges. For example, the molecular weights of the gelling agent can be about 1 kDa to about 1,000 kDa, about 10 kDa to about 1,000 kDa, about 100 kDa to about 1,000 kDa, or about 50 kDa to about 500 kDa.

Examples of thickening agents include lanolin, hard paraffin, liquid paraffin, white petrolatum, soft yellow paraffin or soft white paraffin, white beeswax, yellow bees wax, sorbitan monooleate, petroleum, propolis (propoleum), cetostearyl alcohol, cetyl alcohol, dimethicones, emulsifying waxes, microcrystalline wax, oleyl alcohol and stearyl alcohol.

A gelling agent or thickening agent can be present in a formulation at about 0.05 weight percent to about 20 weight percent, typically about 0.1 weight percent to about 10 weight percent, about 0.1 weight percent to about 5 weight percent, about 0.5 weight percent to about 2 weight percent, about 0.8 weight percent to about 2 weight percent, or about 1-1.5 weight percent. In one embodiment, the composition comprises 0.5 weight percent to 15 weight percent, 1 weight percent to 10 weight percent, or 2 weight percent to 10 weight percent, of one or more thickening agents or gelling agents. In other embodiments, the gelling agent is present in an amount of about 5 to about 10 weight percent, or about 5 to about 15 weight percent, based on the total weight of the composition.

One or more gelling agents or thickening agents may be included in a single formulation. Such agents can be employed with liquid carriers to form spreadable gels, pastes, ointments, soaps, and the like, for application directly to the skin of the user.

### Dosage Forms

### Solutions and Dispersions

Solutions of an active agent or its salts can be prepared in water, optionally mixed with a nontoxic surfactant. Dispersions can be prepared in glycerol, liquid PEGs, triacetin, or in a pharmaceutically acceptable oil such as emu oil, or mixtures thereof. Under ordinary conditions of storage and use, preparations may contain a preservative to prevent the growth of microorganisms.

Pharmaceutical dosage forms can include sterile aqueous solutions or dispersions including the active ingredient adapted for the extemporaneous preparation of sterile solutions or dispersions, optionally encapsulated in liposomes. The ultimate dosage form should be fluid and stable under the conditions of manufacture and storage. The liquid carrier or vehicle can be a solvent or liquid dispersion medium including, for example, water, ethanol, a polyol (for example, glycerol, propylene glycol, liquid PEGs, and the like), vegetable oils, emu oil, nontoxic glyceryl esters, and suitable mixtures thereof. The proper fluidity of the composition can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it is preferable to include isotonic agents, for example, sugars, buffers, or sodium chloride. Prolonged absorption of the compositions can be brought about by agents delaying absorption, for example, aluminum monostearate and/or gelatin.

Solutions can be prepared by incorporating the active in a desired amount in the appropriate solvent or oil with various other ingredients enumerated herein, as desired, followed by optional filter sterilization. For powders used in the preparation of solutions, methods of preparation can include vacuum drying and freeze-drying techniques, which yield a powder of the active plus any additional desired ingredient present in the prepared solutions.

### Gels

Gels are clear, sticky, jelly-like semisolids or solids prepared from high molecular weight polymers in an aqueous or alcoholic base. Alcoholic gels are often drying and cooling. Non-alcoholic gels are more lubricating. Gels or jellies can be produced using a suitable gelling agent including, but not limited to, gelatin, tragacanth, a carbomer, or a cellulose derivative and may include glycerol as a humectant, an emollient, and/or a preservative. In some embodiments, gel formulations will include the same or similar ingredients as a solution or dispersion, with the addition of a gelling agent.

The gel can include a nonionic copolymer gelling agent. In one embodiment, the gelling agent is a nonionic polyoxyethylene-polyoxypropylene copolymer gel, for example, a Pluronic gel such as Pluronic® F-127 surfactant (BASF Corp.), to provide a pluronic gel-based formulation. This gel can be advantageous because it is a liquid at low temperatures but rapidly sets at physiological temperatures, which confines the release of the agent to the site of application or immediately adjacent that site. Other formulations can be CMC-based formulations, HMC-based formulations, HPC-based formulations, or HPMC-based formulations, and the like.

### Creams

Creams are viscous liquids or semisolid emulsions, either oil-in-water or water-in-oil emulsions. Cream bases are water-washable, and include an oil phase, an emulsifier, and an aqueous-phase. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax.

Suitable properties of the cream base include the ability to modify the viscosity of the emulsion and both physical and chemical stability over a wide range of pH. The water soluble or miscible cream base may contain a preservative system and may also be buffered to maintain an acceptable physiological pH.

The oil phase, also called the “internal” phase, generally includes petrolatum and a fatty alcohol such as cetyl or stearyl alcohol. The aqueous phase usually, although not necessarily, exceeds the oil phase in volume, and generally contains a humectant (a substance, such as glycerin, sorbitol, sorbitan sesquioleate, or urea, that absorbs or helps another substance retain moisture).

The emulsifier in a cream formulation is generally a nonionic, anionic, cationic, or amphoteric surfactant. Examples of emulsifiers include, but are not limited to, fatty alcohol polyoxyethylene ether (Peregal A-20), stearates such as polyoxylstearate (Softener SG), glyceryl stearate and pegylated forms of glyceryl stearate such as PEG-5 glyceryl stearate, PEG-100 stearate, cetyl alcohol, lecithin, hydrogenated lecithin, lanolin, squalene, dithranol, or a combination thereof.

In several embodiments, the primary emulsifier includes dimethicone copolyol, sorbitan sesquioleate, cyclopentasiloxane, PPG/PEG-18/18 dimethicone, dimethiconol, or any combination thereof. In some embodiments, the dimethicone copolyol is selected from Dimethicone PEG-8 Adipate, Dimethicone PEG-8 Benzoate, Dimethicone PEG-7 Phosphate, Dimethicone PEG-10 Phosphate, Dimethicone PEG/PPG-20/23 Benzoate, Dimethicone PEG/PPG-7/4 Phosphate, Dimethicone PEG/PPG-12/4 Phosphate, PEG-3 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-6/11 Dimethicone, PEG/PPG-8/14 Dimethicone, PEG/PPG-14/4 Dimethicone, PEG/PPG-15/15 Dimethicone, PEG/PPG-16/2 Dimethicone, PEG/PPG-17/18 Dimethicone, PEG/PPG-18/18, Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone, PEG/PPG-27/27 Dimethicone, Cetyl PEG/PPG-10/1 Dimethicone, bis-PEG/PPG-14/14 Dimethicone, Lauryl PEG-10 Tris(Trimethylsiloxy)silylethyl Dimethicone, or any combination thereof. In one or more embodiments, the dimethicone copolyol is present in an amount of about 0.80 to about 1.10 weight percent based on the total weight of the composition.

Oil-phase ingredients can include, but are not limited to, dimethicone, dimethiconol, cyclomethicone, diisopropyl adipate, cetyl alcohol, stearyl alcohol, paraffin, petrolatum, almond oil, stearic acid, or a combination thereof. In particular aspects, aqueous ingredients can include, but are not limited to, purified water, glycerol (glycerin), propylene glycol, ethyl paraben, a humectant, or a combination thereof. In various embodiments, dimethicone and/or dimethiconol are skin-conditioning agents. In several embodiments, the dimethicone and/or dimethiconol is present in an amount of about 0.70 to about 1.5 weight percent based on the total weight of the composition.

In some embodiments, the cream further includes one or more film formers including but not limited to polyglycerylmethacrylate, acrylates/C₁₀-C₃₀ alkyl acrylate cross-polymers; antioxidants including but not limiting to tocopheryl acetate; preservatives including but not limited to phenoxyethanol, benzyl alcohol; other additives including but not limited to dicaprylyl ether, disodium EDTA, sodium hydroxide, and lactic acid.

In one embodiment, the cream includes purified water, polyglycerylmethacrylate, propyleneglycol, petrolatum, dicaprylyl ether, PEG-5 glyceryl stearate, glycerin, dimethicone, dimethiconol, cetylalcohol, sweet almond oil, acrylates/C₁₀-C₃₀ alkyl acrylate cross-polymers, tocopheryl acetate, phenoxyethanol, benzyl alcohol, disodium EDTA, sodium hydroxide, lactic acid, or any combination thereof. In another embodiment, the cream can include glycerol, light liquid paraffin, soft white paraffin, dimethicone, squalane, methyl hydroxybenzoate, dichlorobenzyl alcohol, or any combination thereof.

### Ointments

Ointments are semisolid preparations that include the active incorporated into a fatty, waxy, or synthetic base. Ointments are typically based on petrolatum or other petroleum derivatives. The specific ointment base to be used, as will be appreciated by those skilled in the art, is one that will provide for suitable drug delivery and other desired characteristics such as emolliency or the like. As with other carriers or vehicles, an ointment base is typically inert, stable, non-irritating and non-sensitizing. Ointment bases may be generally grouped in four classes: oleaginous bases, emulsifiable bases, emulsion bases, and water-soluble bases. Oleaginous ointment bases can include, for example, vegetable oils, fats obtained from animals such as emu oil, and semisolid hydrocarbons obtained from petroleum. Emulsifiable ointment bases, also known as absorbent ointment bases, contain little or no water and can include, for example, hydroxystearin sulfate, anhydrous lanolin, and hydrophilic petrolatum. Emulsion ointment bases are either water-in-oil (W/O) emulsions or oil-in-water (O/W) emulsions, and the oil components can include, for example, cetyl alcohol, glycerylmonostearate, lanolin, and stearic acid. Water-soluble ointment bases can be prepared from PEGs of varying molecular weight.

### Lotions

Lotions are liquid or semiliquid preparations in which solid particles, including the active agent(s), are present in a water or alcohol base. Lotions are usually suspensions of solids, and can include a liquid oily emulsion of the oil-in-water type. Lotions are often desirable formulations because of the ease of applying a more fluid composition. It is generally advantageous for the insoluble matter in a lotion to be finely divided. Lotions will typically contain suspending agents to produce better dispersions as well as compounds useful for localizing and holding the active agent in contact with the skin, e.g., methylcellulose, sodium carboxymethyl-cellulose, or the like.

### Pastes

Pastes are semisolid dosage forms in which the active agent is suspended in a suitable base. Depending on the nature of the base, pastes are divided between fatty pastes or those made from a single-phase aqueous gel. The base in a fatty paste is generally petrolatum, hydrophilic petrolatum, or the like. The pastes made from single-phase aqueous gels generally incorporate CMC or the like as a base.

### Foams

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methyl paraben and propylparaben.

Accordingly, the composition described herein may be formulated for any desired form of topical or transdermal administration, including slow or delayed release preparations. Formulations may include known antioxidants (e.g., vitamin E), buffering agents, lubricants (e.g., synthetic or natural beeswax), sunscreens (e.g., para-aminobenzoic acid), and cosmetic agents (e.g., coloring agents, fragrances, essential oils, moisturizers, or drying agents). The fragrances may include lavender oil, powder scent, jasmine, gardenia oil, Ylang-Ylang oil, green tea oil, or any combination thereof.

An auxiliary agent such as casein, gelatin, albumin, or sodium alginate may also be included in various formulations. Adjuvants such as fragrances and additional antimicrobial agents can be added to optimize the properties for a given use.

Examples of fragrances include Ylang-Ylang oil, lavender oil, powder scent, jasmine, gardenia oil, or green tea oil. In addition, substances such as wetting or emulsifying agents, stabilizing agents, or pH buffering agents, may also be included. When a water-based carrier is used, the composition is typically near a neutral pH (+/−about 1, or 2, pH units).

Further examples of dermatological ingredients and compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the actives described herein in place of other actives.

The compositions described above can be prepared using standard compounding techniques. For example, for a composition that includes magnesium sulfate, or a salt of an active, the active can be triturated to reduce particle size. This suspension can then be combined with other ingredients, such as a fragrance, to provide a therapeutic composition. The suspension can also be combined with other ingredients to form a variety of formulations, such as a gel, a jelly, a cream, anointment, a wax, a lotion, a paste, a foam, or an aerosol. The suspension, or a gel, jelly, cream, ointment, wax, lotion, or paste can also be incorporated into a patch, such as an occlusive patch, to further improve transdermal penetration.

1. A composition for topical or transdermal administration of magnesium sulfate to an erogenous and/or ano-rectal zone of a patient comprising an effective amount of magnesium sulfate, or a pharmaceutically-acceptable salt thereof, and a pharmaceutically-acceptable carrier or excipient.

2. (canceled)

3. The composition of claim 1, further comprising a gelling agent.

4.-5. (canceled)

6. The composition of claim 1, wherein the pharmaceutically-acceptable carrier or excipient, or gelling agent, comprises beeswax, sorbitan monooleate, petroleum, a skin penetration enhancing agent, or any combination thereof.

7.-11. (canceled)

12. A composition for topical or transdermal administration of magnesium sulfate, or a pharmaceutically-acceptable salt thereof, to an erogenous or ano-rectal zone of a patient, the composition comprising an aqueous phase and a silicone phase provided as a water-in-silicone inverse phase emulsion;
wherein the aqueous phase comprises:
magnesium sulfate in an effective amount;
at least one water-soluble carrier;
a humectant or emollient;
at least one preservative; and
water;
wherein the silicone phase comprises:
a water-insoluble carrier;
a skin conditioning agent;
a primary emulsifier; and
a texture enhancer.

13. (canceled)

14. The composition of claim 12, wherein the magnesium sulfate comprises magnesium sulfate heptahydrate.

15. (canceled)

16. The composition of claim 12, wherein the composition comprises a magnesium sulfate concentration that ranges from 10.0 to 25.0 weight percent based on the total weight of the composition.

17. (canceled)

18. The composition of claim 12, wherein the water-soluble carrier is selected from the group consisting of: glycerine, ethylhexyl glycerine, propylene glycol, dipropylene glycol, butylene glycol, ethoxydiglycol, a polyhydric alcohol, and any combination thereof.

19. The composition of claim 12, wherein the primary emulsifer is selected from the group consisting of: a non-ionic alkyl-modified silane adduct of dimethicone, dimethiconol, a hydrophillically modified alkyl fatty acid ester of a polyol, and any combination thereof.

20.-38. (canceled)

39. A composition for topical or transdermal administration of magnesium sulfate to a patient, the composition comprising:
an aqueous phase comprising:
magnesium sulfate in a concentration that ranges from 10 to 35 weight percent of the total weight of the composition;
at least one water-soluble carrier in a concentration that ranges from 0.5 to 6 weight percent of the total weight of the composition;
a humectant or emollient in a concentration that ranges from 1 to 10 weight percent of the total weight of the composition;
at least one preservative in a concentration that ranges from 0.5 to 5.0 weight percent of the total weight of the composition; and
water in an amount that ranges from 35 to 65 weight percent of the total weight of the composition; and
a silicone phase comprising:
a water-insoluble carrier comprising cyclomethicone in a concentration that ranges from 5 to 35 weight percent of the total weight of the composition;
a skin conditioning agent comprising dimethicone, dimethiconol, or a combination thereof, in a concentration that ranges from 0.7 to 2.0 weight percent of the total weight of the composition;
a primary emulsifier in a concentration that ranges from 0.5 to 5.0 weight percent of the total weight of the composition; and
a texture enhancer comprising C₁₂-C₁₅ alkyl benzoate in a concentration that ranges from 0.5 to 6.0 weight percent of the total weight of the composition;
wherein the aqueous and silicone phases are provided as a water-in-silicone inverse phase emulsion.

40.-42. (canceled)

43. A method of treating erectile dysfunction, premature ejaculation, low libido, or benign prostatic hyperplasia (BPH) in a male patient, or for treating orgasmic sexual dysfunction or low libido in a female patient, comprising topically or transdermally administering to an individual in need an effective dose of the composition of claim 1.

44.-47. (canceled)

48. A method of increasing the size and/or turgidity of an erectile tissue in a male patient, the method comprising topically or transdermally administering to the male patient an effective dose of the composition of claim 1.

49. (canceled)

50. A method of treating orgasmic sexual dysfunction or low libido in a female patient, the method comprising topically or transdermally administering to the female patient an effective dose of the composition of claim 1.

51. (canceled)

52. A method of increasing the size, sensitivity and arousability of an erectile tissue in a female patient, the method comprising topically or transdermally administering to the female patient an effective dose of the composition of claim 1.

53. (canceled)

54. A method of treating orgasmic sexual dysfunction or low libido in a female patient, the method comprising topically or transdermally administering to the female patient an effective dose of the composition of claim 1, to an erogenous zone of the female patient to increase the size, sensitivity and arousability of an erectile tissue in the female patient, resulting in increased sexual arousal.

55. (canceled)

56. The method of claim 43, further comprising relaxing a vascular smooth muscle tissue in an erogenous zone or erectile tissue of the male or female patient.

57. The method of claim 56, wherein relaxing the vascular smooth muscle tissue comprises inhibiting Ca++ influx into the vascular smooth muscle tissue.

58. The method of claim 56, wherein relaxing the vascular smooth muscle tissue comprises activating an adenylate cyclase enzyme in the vascular smooth muscle tissue.

59. The method of claim 56, wherein relaxing the vascular smooth muscle tissue comprises increasing formation of cyclic 3′,5′ adenosine monophosphate (cAMP) in the vascular smooth muscle tissue.

60. The method of claim 56, wherein relaxing the vascular smooth muscle tissue comprises activating a cAMP-dependent protein kinase (PKA) in the vascular smooth muscle tissue.

61. The method of claim 56, wherein the magnesium sulfate or a plurality of Mg++ ions are absorbed through the male or female patient's skin and connective tissue in the erogenous zone or erectile tissue.

62.-80. (canceled)

81. The method of claim 43, wherein the composition is constituted as a condom lubricant, penile lubricant, vaginal lubricant or anal lubricant.

82.-96. (canceled)

97. The method of claim 81, wherein the patient is a female patient, and wherein the composition is applied to an exterior surface or an interior surface of the female patient's genitals.

98.-106. (canceled)

107. A method of treating benign prostatic hyperplasia (BPH) in a male patient, the method comprising administering a topical or transdermal formulation comprising magnesium sulfate, or a pharmaceutically-acceptable salt thereof, to the male patient's penis, ano-rectal or prostate region.

108.-121. (canceled)
